(19) United States
(12) Patent Application Publication
(10) Pub. No.: US 2026/0073565 A1
(43) Pub. Date: Mar. 12, 2026

(54) COMPRESSION OF IMAGE DATA OF A MEDICAL IMAGING DEVICE

(71) Applicant: Siemens Healthineers AG, Forchheim (DE)

(72) Inventor: Johann SUKKAU, Herzogenaurach (DE)

(73) Assignee: Siemens Healthineers AG, Forchheim (DE)

(21) Appl. No.: 19/324,466

(22) Filed: Sep. 10, 2025

(30) Foreign Application Priority Data
Sep. 11, 2024 (DE) 10 2024 208 619.3

(51) Int. Cl. G06T 9/00; G16H 30/40

(52) U.S. Cl. CPC G06T 9/00; G16H 30/40

(57) ABSTRACT

A computer-implemented method for compression of image data of a medical imaging device, comprises: stipulating at least one threshold value for image point values for assignment of image points to useful data and to background data on different sides of the at least one threshold value; creating a data header including information about the compression applied so that, based on the information, a reconstruction of the image data from the compressed image data is possible; reducing the image data to data points belonging to the useful data; storing the image point values of the useful data without the image point values of the background data; and storing location information that allows the location of the data points of the useful data, together with respective image point values, to be reconstructed on at least one computer-readable storage medium.

### CROSS-REFERENCE TO RELATED APPLICATION(S)

The present application claims priority under 35 U.S.C. § 119 to German Patent Application No. 10 2024 208 619.3, filed Sep. 11, 2024, the entire contents of which is incorporated herein by reference.

### FIELD

One or more example embodiments of the present invention relate to a computer-implemented method for compression of image data of a medical imaging device, to a method for recording and storage of image data of a medical imaging device, to a non-transitory computer program product and to a medical imaging device.

### BACKGROUND

In a medical imaging device based on Positron Emission Tomography (PET), gamma quanta are slowed down by material that is located between a PET signal source of the medical imaging device and gamma detectors of the medical imaging device. The point in space at which the electron-positron annihilation takes place and two gamma quanta are emitted in the opposite direction can in particular be referred to as the signal source. According to the current state of the art the gamma detectors can in particular comprise LSO crystals, which function as scintillators in that they convert the gamma quanta into photons, and CMOS photodetectors which convert these photons into electrons. The slowing down of the gamma quanta leads to an attenuation of the PET signal, which in turn has a negative influence of the PET signal quality. This attenuation of the signal is therefore typically to be avoided where possible (in that for example it is ensured that there are no obstacles on the path) or the PET signal is corrected retrospectively. For this Attenuation Correction (AC), so-called μ-maps (attenuation maps) are used, which as a rule comprise image points with image point values at defined positions. These μ-maps are typically available for example for the patient couch and the patient (in the case of PET, PET-CT and MR-PET; CT stands in this case for Computed Tomography, MR stands for Magnetic Resonance or Magnetic Resonance Tomography) and for local MR coils (as a rule only MR-PET). Typically these p-maps are either recorded with a CT (for example couch, coils) or directly with MR (for example patient). After being recorded, this data is typically stored as voxel data resolved in three spatial directions.

The quantities of data accumulating here are typically relatively large, for example ranging between 1 and 8 gigabytes, and therefore need correspondingly large amounts of space on a data medium. This can be a problem in particular when data is to be archived and saved with backups, on online servers for example. Thus large quantities of data give rise to additional costs, which typically correlate directly with the size of the data.

### SUMMARY

Independent of the grammatical term usage of a specific person-related term, individuals with male, female or other gender identities should be included within the term.

The inventors have determined that it would be desirable to find an option for reducing the size of corresponding data as much as possible. In the prior art corresponding μ-map data has previously not been compressed. Furthermore, conventional lossless methods for data compression often only produce a slight reduction in the size of the data of corresponding μ-maps.

An object of one or more example embodiments of the present invention is therefore to find an option for reducing the size of μ-map data or to find an improved method for data compression of the μ-map data, preferably without having to take into account a loss of information of the data. Furthermore, it would be desirable in general to find an option for compression or for improved compression of medical image data or at least of some types of medical image data.

At least this object is achieved by methods, non-transitory computer program products, and medical imaging devices as claimed in the independent claims. Further features and advantages emerge from the dependent claims, the description and the attached figures.

In accordance with a first aspect of one or more example embodiments of the present invention, a computer-implemented method for compression of image data of a medical imaging device is provided, wherein the image data comprises image points with image point values at defined positions. The method comprises the following steps:
stipulating at least one threshold value for the image point values for assignment of the image points to useful data and to background data on respective sides of the threshold value;
creating a data header comprising information about the compression applied, so that, based on the information of the data header, a reconstruction of the image data from the compressed data is made possible;
-reducing the image data to the data points belonging to useful data and storage of the image point values of the useful data without the image point values of the background data and also storage of location information, which allows the location of the data points of the useful data together with the respective image point values to be reconstructed, on at least one computer-readable storage medium.

Image data of PET attenuation maps (i.e. μ-maps) is typically multi-dimensional volume files, which is sparsely populated by useful data. This is attributable in particular to the fact that only a (relatively small) part of the space considered is filled with signal-attenuating obstacles, for example a patient couch (in particular in lightweight construction with a large proportion of air in the frame). The inventive method is especially well suited for such μ-maps or for other medical image data that is sparsely populated with useful data. Advantageously with the inventive method a compression of the data can be achieved, with which the image data (for example the μ-maps of a MR-PET scanner) can be stored on a data medium and read back again from said medium very easily, rapidly and in a space-saving manner. In particular the costs of archiving the image data can be lowered with this. Also the reading in of the image data from a data medium into the main memory of a user PC (Personal Computer) can be undertaken much more quickly. A sparse population with useful data is to be understood in this case as a not insignificant part of data points not being useful data (i.e they are background data). In particular sparsely populated can mean that a maximum of 85%, preferably a maximum of 50%, especially preferably a maximum of 25%, quite especially preferably a maximum of 10% of the image points is background data. The larger the share of useful data in the overall image data is, the more effectively the compression with the inventive method can be. The proportion of useful data up to which a compression can still be achieved effectively can in particular depend on the concrete implementation of the respective form of embodiment of the inventive method. With an especially preferred form of embodiment, as described herein, with a proportion of over 80% of useful data, an effective compression of the image data can still be achieved.

The inventive method is a computer-implemented method. The method can for example be carried out directly on a control unit of the medical imaging device. The method can however also be carried out externally from the medical imaging device. The image data in such cases can generally be any kind of image data, which comprises useful data and background data. Background data can in particular be image points that represent an essentially empty space or a space with an essentially zero signal or an image point value lying below a minimum threshold value. Preferably there can be provision for at least 15% preferably at least 50%, especially preferably at least 75%, quite especially preferably at least 90%, of the image points of the image data not to be useful data. The image data can be image data of an attenuation map. The image data can in particular be image data of a PET attenuation map (μ-map). The medical imaging device can in particular be a medical imaging device based on Positron Emission Tomography (PET). For example the medical imaging device can be or can comprise a PET scanner, an MR-PET (Magnetic Resonance PET) and/or a PET-CT (PET Computed Tomograph). An MR-PET is a hybrid device consisting of a PET and MRT (Magnet Resonance Tomograph). A PET-CT is a combined PET Computed Tomography device. Optionally the image data can also be other image data of a medical imaging device that has useful data and background data.

The image data can preferably be multi-dimensional image data, in particular volume image data. The image points can in particular be pixels or voxels. The image point values can in particular be pixel values or voxel values. For example the positions of the image points can be defined via coordinates. For example the coordinates of the image points can be specified by whole number indices, which uniquely specify their position in the image, for example in an image volume.

In accordance with one or more example embodiments of the present invention, at least one threshold value is stipulated for the image point values. The threshold value can apply for the entire volume of the image data, i.e. in particular can be a stipulated threshold value. As an alternative the threshold value can apply locally for a part area of the volume and/or be location-independent. A number of threshold values can be provided for example, which are each assigned to a part area. A threshold value that applies locally for a part area or is different for various part areas can be referred to as an adaptive threshold value. Adaptive threshold values are sensible for example when the boundary between useful data and background data depends on the position of the image points, in particular voxels in the volume of the image data. This can be the case for example with brightness fluctuations in a video stream. The image points can thus be assigned to useful data and to background data. In particular, background data can be image points of which the image point values represent a background of the image data, for example air. The threshold value can be a previously stipulated threshold value. For example the threshold value can be stipulated as a function of the type of image data. For example the threshold value can be determined in a calibration method. A correspondingly stipulated threshold value can then for example always be used in the future for the same type of image data of the same medical imaging device or always be stipulated as the same value. In particular the threshold value can be stipulated by retrieval of the corresponding known threshold value. Optionally the threshold value can also be set or stipulated with aid of known prior knowledge. For example it is typically known which values occur in PET attenuation maps and what these values represent. Accordingly for PET attenuation maps the threshold value can be stipulated so that image point values that correspond to an empty space or to air are assigned as background data.

Furthermore a data header is created, which comprises information about the compression applied. With the aid of the data header the image data compressed with this method can be reconstructed again. The data header comprises information about the compression applied. For example an indicator can be stored in the data header as to the form of embodiment of this method for compression that has been used. Information for decrypting the compressed image data can be stored in the data header. For example the dimensions of the image data can be held in the data header. In addition, optionally assigned spatial axes and/or a position of the image data in global coordinates can be held in the data header. A position of the image data in global coordinates can specify where the image data was recorded, for example in relation to the position of the medical imaging device. Optionally information about the object that is shown by the image data will be provided in the data header. For example the information “patient couch” or “body coil #2” can be stored in the data header.

The image data is reduced to data points belonging to the useful data and the image point values of the useful data are stored without the image point values of the background data. Thus, for sparsely populated image data, a reduction in the need for storage compared to the prior art can be achieved, in that the image points of the background data are no longer explicitly stored. In addition location information is stored, which allows the location of the image points of the useful data together with respective image point values to be reconstructed. There can be provision for at least one item of location information and/or at least one image point value of at least one image point, together with a control command that describes the compression, to be stored in at least one shared byte. The image point values of the useful data and/or the location information are stored in particular on a computer-readable storage medium or a data medium. The storage medium can be a local or a remote storage medium. The storage medium can for example be a hard disk, an SSD, a flash memory, an online server, or another suitable storage medium. The computer-readable storage medium can be a non-volatile storage medium. The image point values of the useful data and/or the location information can in particular be stored permanently on the computer-readable storage medium.

In accordance with one form of embodiment, during the reduction of the image data the image point values of the useful data are each stored together with a coordinate position. The compression in accordance with this form of embodiment can also be referred to as compression in accordance with a point cloud encoding. In particular the image point values of the useful data can be stored as a list of image points, wherein each image point is defined as a tuple comprising the image point value and the position. Thus, with sparsely occupied image data, a marked reduction in storage requirements can be made possible. The coordinate position can be defined based on a volume grid for example.

In accordance with one form of embodiment, a start point and a linear path are defined for the reduction of the image data, which runs through the image points of the image data, wherein, during reduction of the image data, image point values of directly adjacent image points of the useful data along the linear path are stored without location information and in a defined order and wherein, in the case of gaps between image points of the useful data along the linear path, at least one item of location information is stored in each case from which the gap can be reconstructed. The location information from which the gap can be reconstructed can comprise coordinate information. The location information from which the gap can be reconstructed can in particular describe at least one coordinate changed compared to the last image point, in particular all coordinates of a coordinate position of the next image point on the linear path after the gap changed compared to the last image point. Preferably, within the framework of the location information, only the coordinates of the coordinate position are stored that have changed compared to the coordinates of the last voxel on the linear path. Advantageously it can be possible with this form of embodiment to store fewer individual coordinates overall and thus to reduce the storage space required. The storage of fewer individual coordinates is in particular made possible by the defined linear path. The linear path can be defined in the data header. For example a start point and a course of the linear path can be defined in the data header. Thus, precisely when many adjacent image points are to be assigned to the useful data, once again a significant reduction in the storage can be made possible. Typically adjacent image points are to be expected in corresponding image data when the image data relates to objects that belong together. There can be provision, during the compression or the reduction of the image data, to move along the linear path and, in doing so, compare each image point value with the at least one threshold value in order to establish whether the respective image point belongs to the useful data. In particular in this case each image point value can be compared with a stipulated or with an adaptive threshold value. Thus it can advantageously be directly established which image points belong to the useful data. There can be provision to store a control command, which describes the compression in each case together with an item of location information and/or together with an image point value. A control command can for example describe that a stored item of information is an item of location information in a specific coordinate direction. A control command can for example describe that a stored item of information is an image point value. For example one control command can be provided in each case for each coordinate direction and one control command to designate the image point values. For example N+1 control commands can be provided for N-dimensional image data. For example 4 control commands can be provided in three-dimensional volume data. There can be provision for the location information to be able to be reconstructed from the gap, and/or for at least one image point value to be stored together with a control command in at least one common byte. For example it can be possible in accordance with this form of embodiment to store a control command in only two bits. Therefore it can be advantageous not to use a separate byte for the control command, but to store the respective location information and/or at least one image point value together with the control command. There can be provision for defining a data packet length, which describes a number of bytes per unit of information. A unit of information can in particular comprise one control command in each case as well as least one item of location information and/or at least one image point value. A data packet length of between 16 bits and 32 bits can be provided for example.

In accordance with one form of embodiment, coordinates of the coordinate system are defined for the reduction of the image data, wherein the linear path is defined in such a way that it runs through the coordinates as nested loops with a first of the coordinates as the innermost loop and a last of the coordinates as the outermost loop. The coordinate system can correspond to a defined coordinate system of the medical imaging device. The coordinate system can for example be a Cartesian coordinate system, in particular with the coordinates X, Y and Z. However another suitable coordinate system, for example a two-dimensional or generally N-dimensional coordinate system or a cylinder coordinate system can also be chosen. Nested loops are in particular to be understood in the sense of the term in programming. For example the linear path can be defined as nested count loops, wherein in particular the coordinate value of the coordinates in the count loops is incremented in steps corresponding to the resolution of the respective coordinates. The coordinate of the innermost loop can be seen as the preferred direction. The preferred direction can in particular correspond to the main longitudinal direction of an object in the image data. Preferably the loops are defined so that a compression is optimized.

In accordance with one form of embodiment, the order of the nested loops is selected in such a way that the number of the gaps along the linear path is minimized and/or that the size of the compressed image data is minimized and/or that the coordinate of the innermost loop comprises the fewest gaps and/or that the coordinate of the innermost loop describes the greatest dimension of the image data. Advantageously the nesting of the loops can thus be selected so that the compression is at its most effective or that the size of the image data is reduced most greatly. For example the nesting can be defined relatively easily by trial and error, in that the compression is applied for a representative example of image data with variable nesting of the loops of the coordinates. Typically a nesting stipulated accordingly can then be used once again in the future since it can be assumed that similar problems favor a corresponding nesting. The selected nesting is preferably held in the data header, in particular in order to make a later reconstruction of the data possible.

In accordance with one form of embodiment, the coordinate selected as the first coordinate for the innermost loop is that coordinate which has, along the direction thereof, the largest number of directly adjacent image points of the useful data. With this measure the compression in particular can be especially efficient.

In accordance with one form of embodiment, the image data, in particular the volume data to be compressed, is image data of an attenuation map, in particular of an attenuation map for a Positron Emission Tomography device. An attenuation map for a Positron Emission Tomography device can be referred to within the framework of this invention as a PET attenuation map. Such an attenuation map is also referred to as a μ-map. Due to the often relatively large amount of background data on such attenuation maps the inventive method is especially well suited to attenuation maps. Background data in attenuation maps is in particular image points at which essentially no attenuation is recorded.

In accordance with one form of embodiment, the image data is image data of at least one hardware component of the medical imaging device, in particular image data of a magnetic resonance tomography hardware component, for example image data of a patient couch, in particular an MR patient couch, a magnetic resonance tomography coil and/or a magnetic resonance tomography phantom.

In accordance with one form of embodiment, the image data is image data of a patient couch. In particular the image data can be a PET attenuation map of a patient couch. For example as a rule most of the image points of a PET attenuation map of a patient couch, seen in cross section, are black or are a recording of air. These image points are then in particular background data. Accordingly as a rule there is only relatively little really relevant useful data, which is relevant for example for an attenuation correction. Thus, due to a typically relatively large number of background data items in the image data of a patient couch, the inventive method can be applied especially effectively to the image data of a patient couch.

In accordance with one form of embodiment, the image data is image data of a patient couch, wherein the first coordinate for the innermost loop is selected as a coordinate parallel to a longitudinal direction of the patient couch. This form of embodiment takes account of the fact that the patient couch in as a rule has the most coherent useful data in the image data in its longitudinal direction. The compression can thus be especially effective.

In accordance with one form of embodiment, during the step of stipulating the at least one threshold value, an input option is provided for a user to set the threshold value, wherein a user input relating to the at least one threshold value is received and the at least one threshold value is set based on the user input. The input option can make it possible to adapt the method individually in accordance with the user's wishes. The input option can make possible an individual optimization of compression tailored to specific image data.

A further aspect of one or more example embodiments of the present invention is a method for recording and storage of image data of a medical imaging device, in particular image data of an attenuation map for a Positron Emission Tomography device, comprising the following steps:
recording the image data with an imaging device;
compression and storage of the image data with a method as described herein.

All advantages and features of the method for compression of image data of a medical imaging device can be transferred by analogy to the method for compression and storage of image data and vice versa. The medical imaging device can for example be or comprise a PET scanner, an MR-PET (Magnetic Resonance PET) and/or a PET-CT (PET Computed Tomograph). For example the image data can be recorded with a Computed Tomograph (CT) or a PET Tomograph or a Magnetic Resonance Tomograph (MRT or MR).

In accordance with one form of embodiment, the image data is first recorded as image data of the imaging device and converted after the recording by the imaging device into an attenuation map for a Positron Emission Tomography device, wherein the image point values of the attenuation map are based in particular on a linear attenuation coefficient. The image data can be a PET attenuation map, which is recorded by CT, PET or MRT or is based on such a recording.

A further aspect of one or more example embodiments of the present invention is a non-transitory computer program product or a non-transitory computer-readable storage medium, comprising commands that, when executed by a computer, cause said computer to carry out the steps of the method as described herein. All advantages and features of the method for compression of image data of a medical imaging device and of the method for recording and storage of image data can be transferred by analogy to the computer program product and vice versa. The computer program product can for example be stored on the computer-readable storage medium, in particular a non-volatile storage medium. The storage medium can for example be a hard disk, an SSD, a flash memory, an online server, etc.

A further aspect of one or more example embodiments of the present invention is a medical imaging system comprising an imaging device, wherein the imaging system is designed to carry out a method as described herein. All advantages and features of the method for compression of image data of a medical imaging device, of the method for recording and storage of image data and of the computer program product can be transferred by analogy to the medical imaging system and vice versa.

A further aspect of one or more example embodiments of the present invention is image data of a medical imaging device, wherein the image data is compressed with a method as described herein. All advantages and features of the method for compression of image data of a medical imaging device, of the method for recording and storage of image data, of the computer program product and of the imaging system can be transferred by analogy to the image data of a medical imaging device and vice versa.

A further aspect of one or more example embodiments of the present invention is a method for reading out image data from compressed data that has been compressed in a accordance with a method described herein. The method comprises the following steps:
reading the data header in order to obtain information about the compression applied; and
reconstructing the image data from the compressed data based on the information about the compression applied, wherein the image data is in particular put back into the uncompressed format.

All advantages and features of the method for compression of image data of a medical imaging device, of the method for recording and storage of image data, of the computer program product, of the imaging system and of the image data of a medical imaging device can be transferred by analogy to the method for reading out image data from compressed Daten and vice versa. information about the compression applied can for example comprise a number of dimensions of the image data and its size. During reconstruction of the image data, the actions used for the compression can be carried out in reverse order. For example a start value of a linear path can be determined, based on which the image data has been compressed, and the image points along the linear path, in particular along nested loops of the linear path, can be read in. In particular the same control commands can be used as are used during compression, in order to make possible an assignment or decryption of the stored compressed data and to write the image values at the correct target position in each case. The can optionally be provision for a subsequent formatting of the image data. For example for the case in which the image data is mage data of a PET attenuation map, there can be formatting that makes an attenuation correction possible. For example the resolution and/or scaling of the image data can be adapted to a resolution needed by interpolation and optionally by multiplanar reconstruction.

All forms of embodiment described herein can be combined with one another unless explicitly specified otherwise.

### BRIEF DESCRIPTION OF THE DRAWINGS

Described below are forms of embodiment that relate to the attached figures.

FIG. 1 shows a slice of a PET attenuation map of patient couch in a transversal section or a cross-section,

FIG. 2 shows a flow diagram of a computer-implemented method for compression of image data of a medical imaging device in accordance with a form of embodiment of the present invention,

FIG. 3 shows a representation of a spatial volume with three-dimensional image data in a coordinate system for illustrating a form of embodiment of this invention,

FIG. 4 shows a diagram in which the size of the image data is plotted as a function of the proportion of useful data in the image data before and after the inventive method in accordance with various forms of embodiment,

FIG. 5 shows a medical imaging system comprising an imaging device in accordance with a form of embodiment of the present invention and

FIG. 6 shows a flow diagram of a method for reading out image data from compressed data in accordance with a form of embodiment of the present invention.

### DETAILED DESCRIPTION

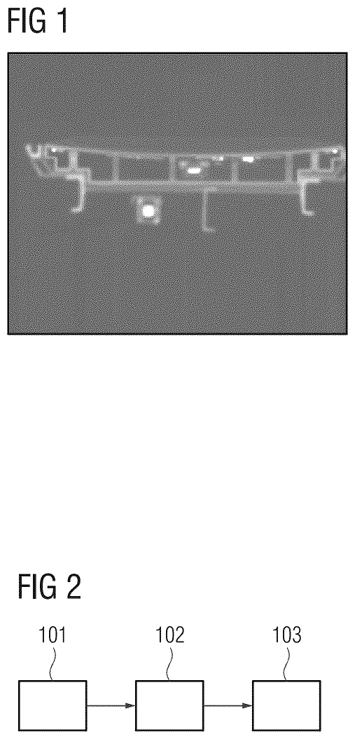

FIG. 1 shows a slice of a PET attenuation map of a patient couch in a transversal section or a cross section. Such an attenuation map can be created for example by a measurement with a computed tomograph. In this case the Hounsfield data of the computed tomograph can be converted according to conversion methods known in the prior art into an attenuation of the PET data, in particular into LAC values. LAC in this case stands for Linear Attenuation Coefficient. LAC values are typically specified in the unit 1/cm. As can be seen in this cross-section, most of the image points, in this case voxels, are black, which means they have the value zero or approximately zero. Shown bright is a composite plastic of the patient couch (dark white) as well as much thicker copper components, such as copper wires, copper screws, etc. (bright white). The white voxels (bright and dark white) correspond to useful data 3. The black voxels correspond to background data. Essentially the useful data 3 is relevant for an attenuation correction, whereby, compared to the total volume, only relatively few voxels actually contain relevant data. The same applies to attenuation maps of MR coils (for example head coil). Also MR coils, although they are likewise voluminous, are only sparsely populated with significant voxel data, i.e. within the framework of one or more example embodiments of the present invention in particular populated by useful data 3. Image data of this type, within the framework of one or more example embodiments of the present invention, can be referred to as sparsely populated image data. The inventive method for compression of the image data can be especially advantageous for sparsely populated image data. In general the inventive method can also be applied generally to sparsely populated image data, in particular sparsely populated multidimensional volume data. In general with such data the voxels can be specified by whole number indices, which uniquely specify a position of the voxels in the volume. For example a position can be described by the coordinates (x1, x2, x3 . . . xn) with xi as index along a volume axis Xi and n the maximum dimension of the volume. For a three-dimensional volume, n=3. Furthermore each image point, or in this example voxel, contains an image point value. The image point value can also be referred to in simple terms as the “value”. The image point value of a voxel can also be referred to as a voxel value. In general the image point value does not necessarily have to be a whole number scalar. For example the image point value can also contain any given complex values (also vectors for example), in particular also in an accuracy/bit depth needed or desired. In the concrete example of FIG. 1 the image point value specifies a strength of the attenuation, which is shown in FIG. 1 by the degree of brightness, so that a greater brightness means a greater attenuation. The image point value can be specified as a function of the position as value (x1, x2, x3 . . . xn). Attenuation maps typically include whole-number, positive intensities with 16-bit resolution as image point values or voxel values. For an attenuation map of the patient couch as can be seen in FIG. 1, with a sampling of 1 mm for example, a volume with a total of 2200×400×600 voxels is produced. In this volume for example each voxel can have 16-bit image point values (corresponds to the accuracy of the intensity value of the attenuation). In this example an uncompressed data size of approx. 1 GB (1056 MB) is produced. Typically such image data is held linearly in the memory sorted according to rows, columns and slices of the volume image data. The position of a voxels can then be specified in the memory as a memory index for the linear memory access, for example in accordance with:

memoryIndex=(((z*VolumeSizeY)+y)*VolumeSizeX+x).

This enable the image point value or value of the voxel to be established, for example in the form

value=muMap[memoryindex],

wherein the position of the voxel in whole-number volume coordinates is (x, y, z). If it is now assumed that only a small part, for example one percent, of the volume data contains useful information, i.e. is useful data 3, then with an inventive method a marked reduction in the storage requirement needed can be achieved.

FIG. 2 shows a flow diagram of a computer-implemented method for compression of image data of a medical imaging device in accordance with a form of embodiment of the present invention. As described by way of example in relation to FIG. 1, the image data contains image points with image point values at defined positions. In a first step 101 at least one threshold value is stipulated for the image point values. The threshold value serves to assign the image points to useful data 3 and to background data on different sides of the threshold value in each case. The threshold value can be stipulated for example by being retrieved from a database, from a memory or from another source in which it is held. As an alternative the threshold value can also be stipulated by a user input for example.

In a further step 102 a data header is created. The data header comprises information about the compression applied, so that, based on the information of the data header, a reconstruction of the compressed data is made possible.

In a further step 103 the image data is reduced to data points belonging to the useful data 3 and the image point values of the useful data 3 are stored without the image point values of the background data. Furthermore location information, which allows the location of the data points of the useful data 3 to be reconstructed together with the respective image point values, is stored, wherein during reduction of the image data, the image point values of the useful data 3 are each stored together with a coordinate position.

In accordance with one form of embodiment, the image point values of the useful data 3 are each stored together with a coordinate position during reduction of the image data. This can also be referred to within the framework of one or more example embodiments of the present invention as point cloud encoding. In particular the image data of the useful data 3 can be stored as a list of voxels, where each voxel is defined as a (position, value) tuple. In the example, which has been explained in relation to FIG. 1, this can mean that 8 bytes of storage space are needed per voxel. These 8 bytes are composed of the three coordinates (x, y, z) with 2×8 bits and the image point value or voxel value with a resolution of 2 bytes (thus 3*2+1*2=8 bytes). With the assumption made above by way of example of 1% useful data 3 and 99% background data (in particular air), this thus produces a reduction of the quantity of data to 42.3 MB and thus to approx. 4% of the original image data of around 1 GB in accordance with this example.

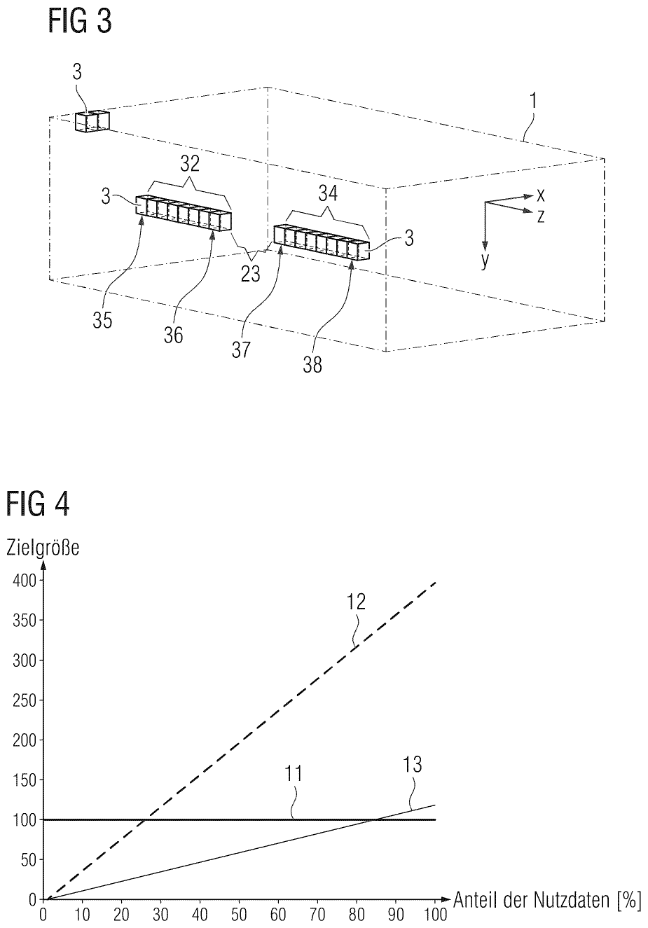

In accordance with one further form of embodiment, a start point and a linear path, which runs through the image points of the image data, are defined for the reduction of the image data. In this case, during reduction of the image data, image point values of directly adjacent image points of the useful data 3 arranged along the linear path are stored without location information and in a defined order and, in the case of gaps between image points of the useful data 3, at least one item of location information is stored in each case along the linear path, from which the gap can be reconstructed. The location information can in particular describe the changed coordinates of the next image point on the linear path after the gap. In this case coordinates of the coordinate system are preferably defined for the reduction of the image data. The coordinates or the coordinate system are or is in particular held in the data header. The linear path is then defined in such a way that it runs through the coordinates as nested loops with a first of the coordinates as the innermost loop and a last of the coordinates as the outermost loop. The loops are preferably chosen so that the compression is maximal. In the example from FIG. 1 the coordinate of the innermost loop can preferably correspond to the longitudinal direction of the patient couch, i.e. in particular direction that runs into the image plane. The longitudinal direction of the patient couch is often defined in medical imaging devices as the z direction. The reduction of the image data in accordance with this form of embodiment will be explained in greater detail in relation to FIG. 3 by way of example and using extracts.

FIG. 3 shows a representation of a spatial volume 1 with three-dimensional image data in a coordinate system with the coordinates x, y, and z. The image data shown here can be considered as a simplified example for image data that has been recorded in a suitable imaging modality (for example computed tomography or magnetic resonance tomography). In accordance with one or more example embodiments of the present invention, a threshold value for the image point values (in this example voxel values) is defined, which separates useful data 3 from background data. The useful data 3 of the image data is shown by small cubes. It can be seen that in this example only a relatively small part of the image data is useful data 3. The image data is consequently sparsely populated. Positions in the spatial volume 1 at which there is no useful data 3 are accordingly occupied by background data, which in this diagram corresponds to the empty space. It can furthermore be seen that a majority of the useful data 3 lies on a beam in the direction of the z axis. For this reason the z coordinate is defined here as the innermost loop. For example the x coordinate can further be set as the middle loop and the y coordinate as the outermost loop. The information about which coordinates are used and which coordinate is used as innermost loop, middle loop and outer loop is stored in the data header of the compressed image data. Further information, such as the global coordinates of the image data for example, can likewise be stored in the data header. For compression of the data all image points (in this example voxels) of the spatial volume 1 of the image data are now visited by the nested loops (in particular count loops or “For loops”) of the coordinates x, y, z, i.e. along a linear path through the spatial volume, the voxel value is compared in each case with the threshold value and the voxels are assigned to useful data 3 or background data. The object in this case is to obtain a list with voxel values (in general: image point values) with an additional coordinate encoding, which serves as location information for the voxel values. The coordinate encoding can be provided in this example by three-dimensional data with 2 bits, which can function in particular as control commands. For example the following states can be described with the two bits:
Bit 00: “SAVE_Value”—SAVE_Value is called when the voxel value is not zero, so that a voxel is thus present that is counted as useful data 3. The corresponding voxel value is then stored behind these bits. This can for example take the form SAVE_Value(VoxelValue_i). Implicitly there is moreover provision to move onwards on the linear path after these bits to the next voxel. No coordinate is explicitly stored for this. A stored sequence of a number of these control commands with associated voxel values after one another, thus means that a number of voxels after one another belong to the useful data 3. If the next voxel on the linear path does not belong to the useful data 3, then the method continues on the linear path to the next voxel that belongs to the useful data 3. The following three bits are then used in addition.
Bit 01: “SAVE_X”—SAVE_X is called when the x coordinate changes or has changed and the voxel value is not zero. Behind these bits the x coordinate of the respective voxel is stored. This can for example have the form SAVE_X(PositionValue_i_X). In other words these bits specify the next x position of the useful data 3 on the linear path, provided the corresponding voxel has an x coordinate other than the last voxel of the useful data 3 on the linear path.
Bit 10: “SAVE_Y”—SAVE_Y is called when the y coordinate changes and the voxel value is not zero. The y coordinate of the respective voxel is stored behind these bits. This can for example have the form SAVE_Y(PositionValue_i_Y). In other words these bits specify the next y position of the useful data 3 on the linear path, provided the corresponding voxel has a y coordinate other than the last voxel of the useful data 3 on the linear path.
Bit 11: “SAVE_Z”—SAVE_Z is called when the Z coordinate changes and the voxel value is not zero. The z coordinate of the respective voxel is stored behind these bits. This can for example have the form SAVE_Z(Positionvalue_i_Z). In other words these bits specify the next z position of the useful data 3 on the linear path, provided the corresponding voxel has a z coordinate other than the last voxel of the useful data 3 on the linear path.

With these bits the compressed data can be written into a linear, binary stream. For example the linear path can begin the origin of the coordinate system set, position (0,0,0) top left. If there is a voxel of the useful data 3 at this position, then the control command or the corresponding bits “SAVE_Value” is/are applied. In the example of FIG. 3, the origin of the coordinate system is not a voxel of the useful data 3 and there is thus a move further along the linear path. This is initially undertaken along the z coordinates (innermost loop) and then along the outer loops, until a voxel of the useful data 3 is reached. Its position is then stipulated by the bits SAVE_X, SAVE_Y and SAVE_Z and its voxel value is stipulated by the bits “SAVE VALUE”. Thus the gap consisting of background data is skipped and only the position and the voxel value of the next voxel with useful data 3 is stored again. These steps are repeated until such time as all voxel values of the useful data 3 have been copied into the destination file.

In the following example, based on FIG. 3, it is assumed that the voxels of the useful data 3 all equally have a voxel value of 128. Moreover it is assumed that, on writing into the destination file, we have just arrived at the beginning of the first section 32 of the stream of useful data 3 in the z direction. In other words we have just arrived at the first voxel 35 of the stream of useful data 3. The storage of sections 32, 34 of the stream of useful data 3 is now described by way of example. The first voxel 35 of the stream is to be at the coordinate position (x=20, y=30, z=0). Since after the previous voxel of the useful data 3, all coordinates are changed, first of all the position of the voxel 35 is stored with the control commands SAVE_X(20), SAVE_Y(30), SAVE_Z(0). Moreover the voxel value of the voxel 35 is stored with SAVE_Value(128). Since the next voxel of the useful data 3 is directly adjacent on the linear path, its voxel value is also stored as the next value with SAVE_Value(128). Thus the method proceeds with the following voxel values until the last voxel 36 of this coherent section 32 is reached. The gap 23 with background data, at which the voxels have the voxel value zero, is then skipped and the z coordinate of the first voxel 37 of the second section 34 with useful data 3 of this z stream is stored with SAVE_Z(11). The first voxel 37 is thus located at the z position 11. The other coordinates (x, y) are not explicitly stored here since they have not changed. Beginning with the first voxel 37 of this section 34 up to the last voxel 38 of this section the voxel values of 128 of these voxels are stored with the control command SAVE-value (128). These can then be stored in the destination file in accordance with the following scheme:

SAVE_X(20), SAVE_Y(30), SAVE_Z(0), SAVE_Value(128), SAVE_Value(128), SAVE_Value(128), SAVE_Value(128), SAVE_Value(128), SAVE_Value(128), SAVE_Value(128), SAVE_Value(128), SAVE_Z(11), SAVE_Value(128), SAVE_Value(128), SAVE_Value(128), SAVE_Value(128), SAVE_Value(128), SAVE_Value(128), SAVE_Value(128), SAVE_Value(128)

Expressed in bits (with the bits “b01”, “b10”, “b11” and “b00”) this section could appear as follows for example:

b01 20 b10 30 b11 0 b00 128 b00 128 b00 128 b00 128 b00 128 b00 128 b00 128 b00 128 b11 11 b00 128 b00 128 b00 128 b00 128 b00 128 b00 128 b00 128 b00 128

Preferably the coordinates and the image point values are stored together with the respective control command in at least one shared byte of a data medium. In accordance with the example specified here, individual control commands only need one storage location of two bits on the data medium. Therefore it can be advantageous not to use a separate byte for the control command, but to store the respective location information the respective image point value jointly with the control command. This enables the compression rate to be further enhanced. For example a data packet length (number of bytes) can be defined, which applies in particular for all information units in a destination data stream. Starting for example from a data packet length of 2 bytes (corresponding to 16 bits), 14 bits can be provided for the useful data and/or for the X, Y-Z coordinates. The remaining two bits are intended for the respective control command, with which it can be distinguished whether this data packet involves an X, Y, Z coordinate or an image data value. Other data packet lengths are also conceivable, for example data packets with a 32-bit length are conceivable, wherein 30 bits are provided for the coordinates or image values of the useful data and 2 bits for the respective control command.

To read the compressed files, corresponding steps can be applied to the method for compression, wherein the destination and the source are swapped. Thus the data can be read out from the compressed file and transferred into the uncompressed format, in that the same linear path is followed. In this case, first the data header is read, and the number of dimensions and their size are transferred. Then the pointer position is set to the start value (0, 0, 0). Then, in the N nested loops (in this example case N=3, and z is the inner loop) all image points (in this example voxels) are read. The SAVE_X/Y/Z commands stored before each image point value or voxel value are used in order to shift the position in the whole-number spatial volume 1. After this the following image point value or voxel value is written into the current destination position. For example there can be provision, in the case of a PET attenuation map or μ-map subsequently to carry out formatting of the three-dimensional image data, in order to make possible an attenuation correction in accordance with a standard format. For example the resolution and/or scaling of the image data can be adapted by interpolation and where necessary by multiplanar reconstruction (MPR) to the required resolution.

FIG. 4 shows a diagram in which the size of the image data is plotted as a function of the proportion of useful data 3 in the image data and according to the inventive method in accordance with various forms of embodiment. A line 11 tracing the size of uncompressed image data, the line 12 tracing the size with a point cloud encoding and the line 13 tracing the size for the compression as described in relation to FIG. 3 are shown. The uncompressed data in this case has a constant course 11, since here no distinction is made between useful data 3 and background data and all image points are handled in the same way. It is shown that compression in accordance with point cloud encoding can be advantageous in this example as soon as the proportion of the useful data 3 is lower than around 278. For a greater proportion of useful data 3, this encoding needs more memory however than in the uncompressed format. By contrast, the compression based on a linear path, as described in relation to FIG. 3, can make possible an even better reduction in the target size. In particular up to a proportion of useful data 3 of around 85% an advantage can be obtained. The inventive method for compression is thus well suited to reducing the storage size of sparsely populated files (such as for example μ-maps or PET attenuation maps, which typically have a proportion of useful data 3 in the range of 3% to 10%).

FIG. 5 shows a medical imaging system comprising an imaging device 4 in accordance with a form of embodiment of the present invention. The imaging device 3 can for example be an MR-PET or a PET-CT. The imaging system is designed to carry out a method for compression of image data as described herein, for example described with reference to FIG. 2 and/or FIG. 3. The method can for example be carried out automatically on a control station 5 of the imaging system.

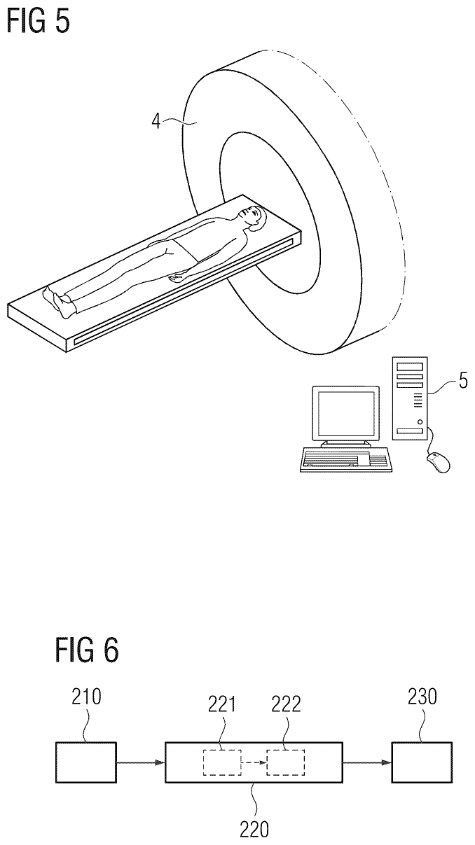

FIG. 6 shows a flow diagram of a method for reading out image data from compressed data that has been compressed in accordance with an inventive method, in accordance with a form of embodiment of the present invention. In a step 210 the data header is read, in order to obtain information about the compression applied. In particular a number of dimensions of the image data and its size can be transferred. In a further step 220 the image data is reconstructed from the compressed data, in particular in that the image data is put back into the uncompressed format. In particular the actions used for the compression can be carried out in the reverse order. For example, starting from a compression as described in relation to FIG. 3, in a step 221 a start value of the linear path can be determined, for example at the coordinate position (0/0/0). In a further step 222, in the nested loops, for example 3 loops, the image points, in particular voxels, can be read in. In this case the same control commands (for example in accordance with the control commands SAVE_Value, SAVE_X, SAVE_X, SAVE_Z described with reference to FIG. 3) are used as were used during compression, in order to make possible an assignment or decryption of the stored compressed data and to write the image values at the correct destination position in each case. Optionally, in a further step 230, there can be formatting of the image data. For example for the case in which the image data image data is a PET attenuation map, there can be formatting which makes an attenuation correction possible. For example the resolution and/or scaling of the image data can be adapted by interpolation and optional multiplanar reconstruction (MPR) to the resolution needed.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for compression of image data of a medical imaging device, the image data including image points with image point values at defined positions, and the computer-implemented method comprising:
stipulating at least one threshold value for the image point values for assignment of the image points to useful data and to background data on different sides of the at least one threshold value;
creating a data header including information about the compression applied so that, based on the information of the data header, a reconstruction of the image data from compressed image data is possible; and
reducing the image data to data points belonging to the useful data;
storing the image point values of the useful data without the image point values of the background data; and
storing location information that allows a location of the data points belonging to the useful data, together with respective image point values, to be reconstructed on at least one computer-readable storage medium.

2. The computer-implemented method as claimed in claim 1,
wherein, a start point and a linear path, which runs through the image points of the image data, are defined for reducing the image data,
wherein, during reduction of the image data, image point values from directly adjacent image points of the useful data along the linear path are stored without location information and in a defined order, and
wherein, in case of gaps between image points of the useful data along the linear path, at least one item of location information is stored as an image point.

3. The computer-implemented method as claimed in claim 2,
wherein coordinates of a coordinate system are defined for reducing the image data, and
wherein the linear path is defined such that the linear path runs through the coordinates as nested loops with a first of the coordinates as an innermost loop and a last of the coordinates as an outermost loop.

4. The computer-implemented method as claimed in claim 3,
wherein an order of the nested loops is selected such that at least one of (i) a number of the gaps between image points of the useful data along the linear path is minimized, (ii) a data size of the compressed image data is minimized, (iii) a coordinate of the innermost loop includes a fewest number of gaps, or (iv) a coordinate of the innermost loop describes a largest dimension of the image data.

5. The computer-implemented method as claimed in claim 3,
wherein a coordinate which has, in a direction thereof, a greatest number of directly adjacent image points of useful data is selected as a first coordinate for the innermost loop.

6. The computer-implemented method as claimed in claim 1,
wherein the image data is image data of at least one hardware component of the medical imaging device of at least one of a magnetic resonance tomography coil or a magnetic resonance tomography phantom.

7. The computer-implemented method as claimed in claim 1, wherein the image data is image data of an attenuation map.

8. A method for recording and storing image data of a medical imaging device, the method comprising:
recording the image data with an imaging device; and
compressing and storing the image data with the computer-implemented method as claimed in claim 1.

9. A non-transitory computer-readable storage medium storing commands that, when executed by a computer, cause said computer to carry out the computer-implemented method as claimed in claim 1.

10. A medical imaging system comprising an imaging device, wherein the medical imaging system is configured to carry out the method as claimed in claim 8.

11. The computer-implemented method of claim 2, wherein the at least one item of location information describes at least one coordinate of a coordinate position on the linear path that is changed compared to a next image point after a gap.

12. The computer-implemented method as claimed in claim 6, wherein the at least one hardware component is a patient couch.

13. The computer-implemented method as claimed in claim 7, wherein the attenuation map is an attenuation map for a Positron Emission Tomography device.

14. The computer-implemented method as claimed in claim 4, wherein a coordinate which has, in a direction thereof, a greatest number of directly adjacent image points of useful data is selected as a first coordinate for the innermost loop.

15. The computer-implemented method as claimed in claim 2,
wherein the image data is image data of at least one hardware component of the medical imaging device of at least one of a magnetic resonance tomography coil or a magnetic resonance tomography phantom.

16. The computer-implemented method as claimed in claim 3,
wherein the image data is image data of at least one hardware component of the medical imaging device of at least one of a magnetic resonance tomography coil or a magnetic resonance tomography phantom.

17. The computer-implemented method as claimed in claim 4,
wherein the image data is image data of at least one hardware component of the medical imaging device of at least one of a magnetic resonance tomography coil or a magnetic resonance tomography phantom.

18. The computer-implemented method as claimed in claim 2, wherein the image data is image data of an attenuation map.

19. The computer-implemented method as claimed in claim 3, wherein the image data is image data of an attenuation map.

20. The computer-implemented method as claimed in claim 4, wherein the image data is image data of an attenuation map.
